(19) United States
(12) Patent Application Publication
(10) Pub. No.: US 2014/0004092 A1
(43) Pub. Date: Jan. 2, 2014

(54) Cancer tumor encapsulation procedure

(71) Applicant: Emil Anghel, Hartford, CT (US)

(72) Inventor: Emil Anghel, Hartford, CT (US)

(21) Appl. No.: 13/917,962

(22) Filed: Jun. 14, 2013

(51) Int. Cl. A61K 38/43

(52) U.S. Cl. CPC A61K 38/43
USPC 424/94.1

(57) ABSTRACT

A cancer therapy and cure based on a painless, safe, effective, inexpensive and free of any side effects procedure aiming to encapsulate malignant tumors via a permanent resilient, thin and malleable shield forcing the cancerous cells contained in them to die and stop them to multiply, or migrate to other parts of the body, or create metastasis.

### BACKGROUND OF THE INVENTION

This disclosure is in the field of medicine, oncology specialty. Specifically, it refers to a new cancer therapy based on a procedure (or method) aiming to encapsulate malignant tumors that would thus become completely and permanently isolated inside the human body. As a result, the cancerous cells confined in tumors will die and they won't be able to multiply anymore, migrate to other parts of the body or create metastasis. The procedure applies primarily to any delineated malignant tumor situated inside the human body.

### BRIEF SUMMARY OF THE INVENTION

Cancerous cells multiply uncontrollably and in order to survive they need to be connected with the human body for having supplied oxygen, feeding and bloodstream. All known traditional cancer treatments aim killing directly the cancerous cells via surgical removal of the tumor(s) if operable or gradually via therapies administrated in sessions. Although some of the existent treatments are successful in the eradication of the malignant tumors, they do not offer any guarantee against possible recurrence of the cancer as some remaining cancerous cells located in the adjacent area of the primary growth could change their status from dormant to active and multiply again in the future. As opposed to current cancer therapies, the procedure disclosed herein deals with the tumor itself and not with its cancerous cells. The tumor is left in place and suppressed as a whole via a permanent resilient encapsulation shield bound to it similar as completely destroying an army and its connections with one strike and not trying to kill its troops individually, in groups, or gradually. Due to the interruption in the blood supply, the whole tumor will become lifeless including all its cancerous cells. This procedure does not involve surgery, it is easily applicable in one session, painless, safe, and free of any side effects. There are four crucial issues to be studied and solved during experimentation and tests of this procedure, as follows: 1. creating the two substances needed to bond together for forming the encapsulation shield that should work, lack toxicity and not be rejected by the human body; 2. the administration mode for the two substances used for the encapsulation shield; 3. making sure that each one of the two substances can be entirely metabolized and eliminated by the human body in case the procedure fails or must be aborted (the entire quantity for the first substance; the unused quantity for the second substance); 4. making sure that the encapsulation shield will be permanent and will maintain its integrity during the lifetime of the patient.

### DETAILED DESCRIPTION OF THE INVENTION

Before applying this procedure, the patient must be subjected to a conclusive radiological test for determining whether he is a proper candidate for it as this therapy can only be used for delineated tumors with clear boundaries separating it from the surrounding tissues so that both cohesive substances be able to completely seal the tumor via encapsulation shield.

This procedure does not apply to non-tumor cancer diseases (such as blood cancer, skin cancer, other) and tumors that for any reason cannot be reached by the two substances used for forming the encapsulation shield.

This procedure is based on a combination of features and principles from the following fields: chemistry, physics, histology, oncology, computer software, radiology.

In specific terms, the procedure aims primarily: a) to create a strong, yet thin and malleable shield—similar to a membrane—completely covering the tumor and its possible extensions into the adjacent areas resembling a sealed sac or pouch; b) to isolate the tumor from the rest of the human body via cutting off of any connection with the living cells and surrounding tissues, glands, organs, joints, muscles, bones; secondarily, it aims to kill all cancerous cells existent in the tumor as a result of the encapsulation.

The procedure is based on the combined use of two substances—such as special enzymes, resins, or mixt composite suspension liquids—that would bond together for encapsulating the tumor, as follows:

1) co-reactant substance that has to be used first. This substance must meet the following requirements and be able: a) to completely cover the tumor and its possible extensions in the adjacent areas similarly as a contrast substance used in radiological tests; b) to temporarily stop possible expansion breach of the cancerous cells; c) to attract the second co-reactive substance as in a magnetic flux; d) to bond to the second co-reactive substance forming together a resilient outer membrane encapsulating the tumor and all its extensions.

2) co-reactive substance that has to be used second. This substance must meet the following requirements and be able: a) to locate the co-reactant substance via self guidance; b) to bond to the co-reactant substance via a chemical molecular process of adherence similarly as the hardener component of the epoxy glue does; c) to form a strong, thin and malleable membrane (or pellicle) when completely cured.

As previously stated, the resultant encapsulation shield must test negative for toxicity. As a temporary exception, either one of the two substances or both could display a low level of toxicity while induced in the body that should cease to exist once the bonding process ends.

Since the encapsulated tumor will not be subjected to physical strain, motion, or friction, the integrity of the membrane should be permanent during the lifetime of the patient. For safety reason, the encapsulation shield must be designed and tested to permanently: hold without breaking a weight equal to a mass tumor in the range of a few dozens of pounds; withstand without leaking or breaking a substantial inner pressure as produced by a large tumor.

The necessary quantity of the co-reactant substance must be determined based on the total measured surface of the tumor(s) via three-dimensional imagery and computer calculation of the dose so that to completely and uniformly cover the tumor including all secluded areas and irregularities in shape and mass.

The quantity for the co-reactive substance should basically be in the same dose as for the co-reactant substance so that to be a match between them. An additional quantity for this second substance—possibly up to 20%—may be needed due to practical considerations as during the bonding process the co-reactant substance could use a bit more of the co-reactive one for covering some hidden or secluded areas of the tumor.

The thickness of the membrane—estimated to be up to 2 (two) sixteenth of an inch—may be slightly uneven as the natural cohesion process between the particles of the two substances cannot be controlled plus in some hidden or secluded areas the shield could form some minor clots. Important here is that the membrane must completely and uniformly cover the tumor.

Any unused quantity of the additional co-reactive substance should be naturally metabolized and entirely eliminated by the human body.

Both co-reactant and co-reactive substances could be liquids administrated intravenously, powder mixed with water, or suspension fluids ingested orally. Based on the research and lab tests that will be performed, the two substances could also be mixt, such as the co-reactant a liquid intravenously administrated and the co-reactive a composite fluid with oral ingestion.

The administration of the two substances must be performed by specialized medical staff in a hospital, clinic or cancer center with monitoring via computerized system. This is one-time treatment during a single session followed by prompt discharge of the patient.

The procedure should take from about one hour to a few hours depending on the following factors: the size and the number of the tumors (single, or multiple); the quantity of the co-reactant substance and the time required for its administration and settlement around the tumor(s); the time required for the co-reactive substance for its administration, settlement around the tumor(s) and wait time for checking on the initial bonding process.

The procedure must be monitored either partially or totally via three-dimensional imagery and computer software. Partial monitoring would involve two checkups: first one for checking on the status of the settlement of the co-reactant; second one for checking on the status of the settlement of the co-reactive plus if both substances started to bond when the membrane should change its color and solidity appearance. Total monitoring would imply a continuous watch of the procedure from start to finish. Each medical facility should decide on the protocol to be applied for performing this procedure as it will be considered fit.

In the rare occurrence when the first co-reactant substance fails to completely and uniformly cover the tumor, the procedure must be aborted with total interdiction for the administration of the second co-reactive substance (so that the co-reactant substance be eliminated by the human body). The procedure may be repeated later using a slightly higher dosage for the co-reactant substance that could have been insufficient during the original session.

The manufacturer of the two substances must provide their specifications, the estimated time for their complete assimilation, settlement around the tumor(s) and the cure time for the shield—membrane (expected to be up to 24 hours).

The computerized system of the medical facilities must be fitted with software assigning a specific color for each of the two substances, also a different color for the membrane when completely cured, plus a visual message and/or a sound signal prompting that the settlement for each of the two substances was completed.

Excluding any possible human errors in performing the procedure—such as improper dosage for each of the two substances, other—the tumor encapsulation disclosed herein should be totally effective, safe and guaranteed to work as intended.

Since the membrane will cure within 24 hours, a follow-up checkup should be performed 7-10 days later for examining its final status. The computerized system should be able to analyze the encapsulation shield and confirm its integrity and functionality as intended.

For tumor(s) located close to the skin surface—such as in exposed areas (neck, armpits or groin, other)—the medical staff must affix over a sturdy bandage for safety. The protective bandage must be left in place for a minimum of 24 hours or until the follow-up checkup time when it will be removed by the medical personnel.

Upon discharge, the patient must receive a special paperwork containing instructions how to protect the encapsulated tumor(s) so that to allow a minimum 24 hours for the shield to cure. Among them, avoiding any strenuous activity in the area of the tumor, direct pressure, push, friction, shock that might negatively affect the smooth natural cure of the membrane.

In case of inoperable tumors, the major and very important difference in the well-being of the patient will be that they will not increase in size and mass anymore as the cells contained in them will die, they will no longer multiply, nor they will create metastasis.

In case of a gland or organ compromised beyond recuperability due to the large size or mass of the tumor, or due to tumor's considerable destructive effect, the procedure disclosed herein could be deemed ineffective but still worth to be performed since the functions of the affected gland or organ may be restored in time.

The cancer tumor encapsulation procedure disclosed herein could also be upgraded from therapy to cure for malignant tumors in the extent that once all cancerous cells contained in them will be forced to commit suicide or die due to asphyxiation, the risk of cancer recurrence should be inexistent. If specific tests performed on the patient subjected to this procedure will confirm the total inexistence of any cancerous cells in the adjacent area of the tumor(s) that will mean that the therapy disclosed herein is in fact a cure for cancer (malignant) tumors.

1. This medical procedure is safe, painless and free of any side effects.

2. This procedure can be used for any delineated cancer (malignant) tumors situated inside the human body.

3. This procedure can also be used for any other tumors—malignant, benign, inoperable or operable—that can be reached by the two substances forming the encapsulation shield.

4. This procedure is effective since once a malignant tumor is encapsulated its cancerous cells will be forced to die with no possible multiplication anymore, or migration inside the human body, or creating metastasis.

5. This procedure has a permanent character unless the patient will opt for an ulterior surgical removal of the operable encapsulated tumor(s).

6. If properly performed, this procedure shouldn't have any margin of errors.

7. This procedure being performed in one session prevails as time factor over all other cancer therapies involving multiple time-consuming treatment sessions.

8. This procedure is cost effective and inexpensive compared to all other existent cancer therapies in use at present time: surgery, chemo, radiation, immuno, hormone, gene therapy.

9. Following the procedure, the patient may resume his normal activities and be completely safe once the encapsulation shield has cured (usually within 24 hours).

10. This procedure could also be a cure for malignant tumors that once permanently isolated from the human body the cancer associated with them will cease to exist.
